(19) United States
(12) Patent Application Publication
(10) Pub. No.: US 2008/0015427 A1
(43) Pub. Date: Jan. 17, 2008

(54) System and network for remote medical procedures

(76) Inventors: Nathan Kastelein, St. Louis, MO (US); Carlo Pappone, Milano (IT); Richard Green, St. Louis, MO (US)

Correspondence Address: Kevin M. Pumm, Suite 400, 7700 Bonhomme, St. Louis, MO, 63105, US

(21) Appl. No.: 11/480,326

(22) Filed: Jun. 30, 2006

(51) Int. Cl. A61B 5/05

(52) U.S. Cl. 600/410

(57) ABSTRACT

Systems and methods are provided for remotely navigating an elongate medical device in an operating region in a subject's body at a local procedure site, under the control of a user at a remote site. A navigation system having a controller responsive to signals from a local computer is provided. An imaging system displays an image of the operating region on a local display, and a physiology monitoring system displays information about the subject. The system utilizes a local computer for providing instructions from a local user to the navigation system controller, and a remote computer for providing instructions from a remote user to the navigation system controller. A display is provided at the remote site, and a video linking system provides a combined video display at the remote site. The system further includes an audio linking system and a data linking system for providing data communication between the remote and local sites.

### FIELD OF THE INVENTION

The present invention relates to the medical procedures which utilize navigation of medical devices within a subject body, and more specifically to remotely performing medical procedures utilizing navigation of medical devices in a subject's body.

### BACKGROUND OF THE INVENTION

Navigation systems have recently been commercially developed for actuation of medical devices to be steered within a patient's anatomy, from a remote location nearby the patient. An example is the Niobe magnetic navigation system developed and sold by Stereotaxis, Inc. Such a system typically allows for control of the navigation of a minimally interventional device with the help of a Graphical User Interface and user input devices such as a mouse, keyboard, joystick or other form of interface input device.

Variability in the complexity of medical procedures, the level of physician skill and training, and proximity to available facilities all contribute to the difficulty of obtaining expert medical treatment or surgical procedures. Computer technology and enhancements in communications such as fiber-optic wireless transmission means have allowed for worldwide transfer of data as well as accessibility to information. While many businesses have capitalized on such technology and have potential access to consumers anywhere in the world through computers, expert interventional surgical medical services are one exception in this regard.

### SUMMARY OF THE INVENTION

The present invention relates to a system and network for remotely performing various medical procedures. Preferably, the system comprises equipment for performing medical procedures using minimally interventional devices that are navigated through a subject's body. In one embodiment in accordance with the present invention, a network and system are provided for enabling remote actuation of a minimally interventional medical device that is to be guided within a subject body's anatomy, for the purpose of performing various medical procedures. The system comprises a navigation system for controlling the orientation of a medical device

In accordance with one aspect of the present invention, a system and network is provided for enabling remote monitoring of a medical procedure being performed in a patient's body. The system comprises at least one full operator station having a navigation control system for controlling the orientation of a minimally interventional medical device that is to be guided within a subject body's anatomy, and one or more remote operator stations in communication with the at least one full operator station, wherein the medical procedure may be monitored from the one or more remote operator stations. The remote operator station may be a visitor operator station, a passive operator station, an active operator station, or another full operator station.

In another aspect of the present invention, a system is provided for enabling an operator to remotely perform a medical procedure in a patient's body at a remote location. The system comprises at least one full operator station having a navigation control system for controlling the orientation of a minimally interventional medical device that is to be guided within a subject body's anatomy, one or more remote operator stations in communication with the at least one full operator station, wherein the medical procedure may be controlled at least partially by an operator at the one or more remote operator stations. Accordingly, a system and network of operator stations may be provided that provides for both educational training, hands on training through remotely performing procedures in a limited capacity, and full control of a medical procedure from a remote location that may be a great distance from the patient and medical facility where the procedure is being conducted.

In another aspect of the present disclosure, embodiments of a system are provided for navigating an elongate flexible medical device in an operating region in a subject's body at a local procedure site, under the control of a user at a remote site. In one embodiment, the system comprises a local navigation system for selectively orienting the distal end of the elongate medical device in the operating region, the navigation system including a controller responsive to control signals provided from a computer. The system includes a local device advancer for advancing and retracting the device in the operating region, the device advancer including a controller responsive to control signals provided from a computer. At least one local medical imaging system is included for displaying an image of the operating region on a local display, and at least one video imaging system is included for providing video images of the local procedure site on a local display. The system further comprises at least one subject physiology monitoring system for displaying information about the subject's physiology on a local display. The system utilizes a local computer for providing instructions from a local user to the navigation system controller and the advancer controller, and a remote computer for providing instructions from a remote user to the navigation system controller and the advancer controller. A display is provided at the remote site, and a video linking system provides a combined video display on the display at the remote site. The combined video display includes the display of the local display of the at least one local medical imaging system, the local display of the at least one local video imaging system, and the local display of the at least one subject physiology monitoring system. The system further includes an audio linking system for providing two way audio communication between the local procedure site and the remote site. The system includes a data linking system for providing data communication between a computer at the remote site and the navigation system controller and the advancer system controller.

In another aspect of the present disclosure, various embodiments are provided of a method for navigating an elongate medical device in an operating region in a subject's body at a local procedure site, by a user at a remote site. In one embodiment of a method, the method comprises displaying on a display at the remote site a combined video image of one or more images being displayed at the local procedure site. The combined video image may include the local display of at least one local medical imaging system which displays an image of the operating region on a local display, the local display of at least one video imaging system that provides video images of the local procedure site on a local display; and the local display of at least one subject physiology monitoring system that displays information about the subject's physiology on a local display.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an illustration of one embodiment of a system and network for enabling control of minimally interventional medical devices from a remote location to perform various medical procedures;

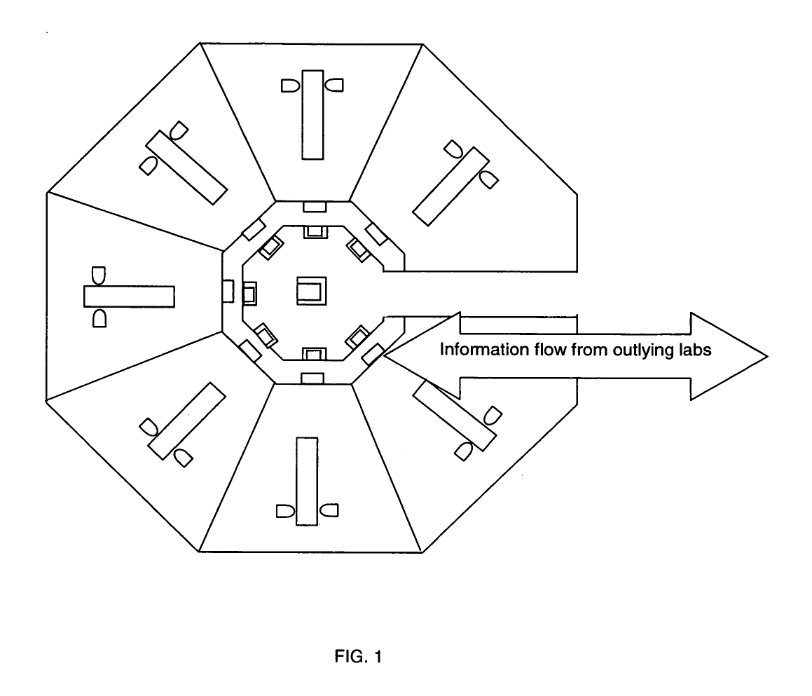

FIG. 2 is an illustration of one embodiment of a system having one or more remote operator stations in communication with a local router;

FIG. 3 is an illustration of one embodiment of a system having one or more visitor operator stations in communication with a full operator station;

FIG. 4 is an illustration of one embodiment of a system having one or more passive operator stations in communication with a full operator station;

FIG. 5 is an illustration of one embodiment of a system having one or more active operator stations in communication with a full operator station;

FIG. 6 is an illustration of one embodiment of a system having one or more other full operator stations in communication with a full operator station;

FIG. 7 is an illustration of a private local network in communication with one or more full operator stations; and

FIG. 8 is an illustration of one embodiment of a system having a satellite communication link for enabling remotely performing a medical procedure at distant locations.

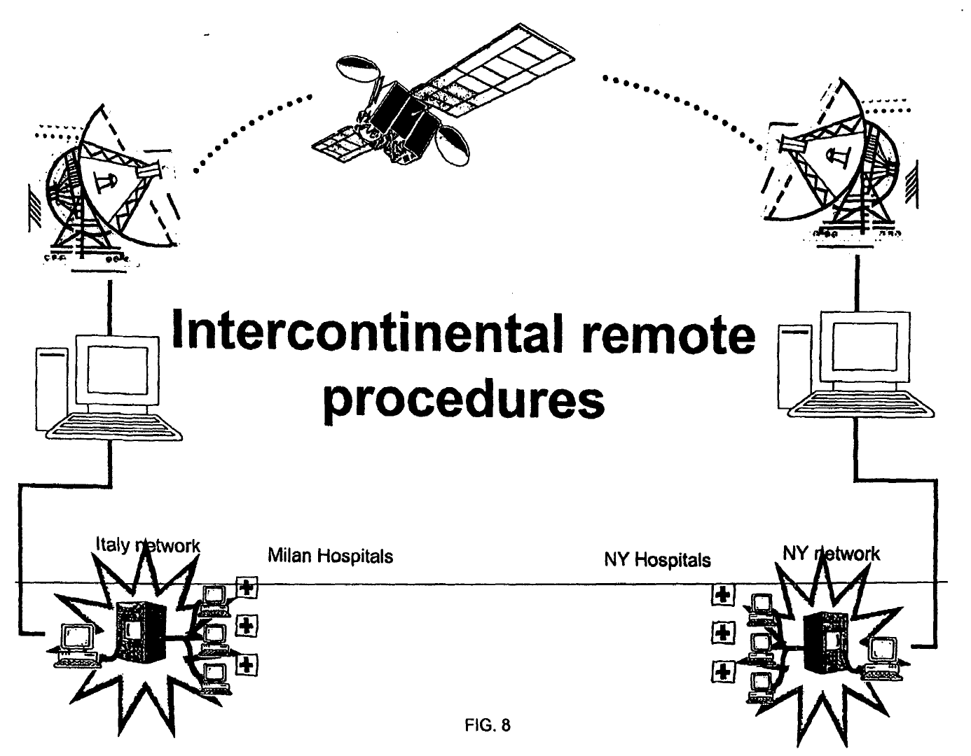

FIG. 9 is a functional diagram of one embodiment of a system for remotely controlling a medical procedure performed on a subject at a local treatment site according to the principles of the present invention.

### DETAILED DESCRIPTION OF THE EMBODIMENTS

The following description of the various embodiments is merely exemplary in nature and is in no way intended to limit the invention, its application, or uses.

The network and system for remotely performing minimally invasive procedures comprises a navigation system for controlling the orientation of a medical device such as a catheter within a patient's body. Navigation systems have been commercially developed recently for actuation of medical devices to be steered within a patient's anatomy, from a remote location nearby the patient. An example is the Niobe magnetic navigation system developed and sold by Stereotaxis, Inc. Such a system typically allows for control of the navigation of a minimally interventional device that is inserted within a patient, with the help of a Graphical User Interface and user input devices such as a mouse, keyboard, or joystick that may be located in a control area near the patient.

The concept of remotely mapping cardiac substrates and remotely delivering therapies to the diseased heart has been recently developed with the advent of Stereotaxis Navigational Systems. Physicians possessing expertise in such navigation systems have performed electrophysiology (EP) mapping of heart tissue, and ablation of supraventricular and ventricular tachyarrhythmias. Moreover, given the special nature of the learning curve of procedures using such medical device navigation systems, there is also the utility of remote learning of EP procedures. In one aspect of the present invention, one embodiment of an integrated network system provides for remotely performing minimally invasive medical procedures on a subject body, remotely delivering or performing treatment of a subject body, and remotely providing instruction for learning the procedures being performed by utilizing a satellite-based telecommunication network and/or a fiber-optic communication network. Expert surgeons can perform medical procedures at a full surgical station with a Stereotaxis Navigation system, which other surgeons in remote locations may monitor or even participate in from a passive station under the supervision of the expert surgeon. Alternatively, an expert surgeon may supervise or even perform a medical procedure being conducted at a full surgical station from a remote passive station, while other surgeons at the full surgical station can watch or assist the expert during the procedure. Passive stations may also be used to rehearse a medical procedure at a remote passive or active station, by using pre-operative images of the subject's body presented on the display console. The surgeon can become familiar with the procedure to be performed, and even practice the procedure in a virtual surgery. In this manner, a surgeon may reliably perform a medical procedure on a patient using a minimally interventional device, such as an electrophysiology catheter, from a remote location using the network and system of the present invention.

Various embodiments of the present invention provide for networking one or more Medical Device Navigational Control Systems used in the fields of cardiac mapping and ablation for SVT and VT and in the CRT applications, to provide for remotely performing electrophysiology mapping of a heart, remotely delivering or performing treatment of a subject body, and remotely providing instruction for learning the procedures. In various embodiments of an integrated network of Medical Device Navigational Control systems, one or more features may be provided, including remotely viewing procedures for training purposes, remotely performing procedures with limited passive control of a System, remotely performing procedures with active control of a system, and Full Control systems that allow either passive or active performing of procedures from a remote location. The system provides for performing remote procedures using Stereotaxis navigation equipment and an integrated network utilizing fiber-optic and satellite communication, for learning and remotely conducting EP procedures including ablation of supraventricular and ventricular tachyarrhythmias and for deliver LV stimulation in the CRT setting. Within the system and network, different kinds of operator stations for remote procedures may be provided as detailed below.

Visitor Station. A visitor station will be equipped with a Navigation system console screen and a selection monitor to connect with other active, passive or full stations to enable remote learning about remotely performed medical procedures. In this way, regional teaching centers could be developed in which to organize teaching sessions. Similarly, during cardiology international congresses, a Visitor Station could be useful for directly showing EP procedures and doing dedicated courses for educating people.

Passive Station. A passive station will be equipped with a Navigation console compatible with a Stereotaxis Navigation system, i.e. fully equipped for conducting remote medical procedures from different sites, as part of a shared EP lab, for example. This passive station could be used both for performing medical procedures and for learning procedures on an animal model of cardiac disease. A passive station is connected to at least one Active Station, and is preferably connected to numerous active stations. Thus, Passive Stations may be utilized for advanced remote learning on animal models and for remotely performing medical procedures on patients at Full Surgical Stations. Moreover, Passive Stations can further include a safety algorithm to ensure patient safety. For example, the algorithm may provide predefined zones in which ablation is excluded (i.e. PVs, His bundle, RBBB, etc) depending on the type of remote procedure. The algorithm may also predefine RF automatic controls, where RF energy is applied for no more than 30-60 sec depending on the type of procedure. The algorithm may further provide automatic Impedance monitoring, automatic signal abatement monitoring, and a one-touch safety key.

Active Station. An active station will be equipped with a Navigation console screen and CardioDrive for remote procedures from that site. Many Active Stations could be connected to the same shared Passive Station. In this way, regional centers with a Full Surgical Station with a Stereotaxis Navigation system can be set up and remotely used from many different local Active Stations.

Full Surgical Station. A full working station with Navigation console screen, Cardiodrive and Stereotaxis Navigation system for incoming and outcoming remote procedures can be installed in few high-trained centers. The Full Stations enable incoming operator-assisted remote procedures from other Passive Stations, or outcoming procedures towards other Active Stations requiring consulting and supervision, and intensive learning towards many Visitor Stations.

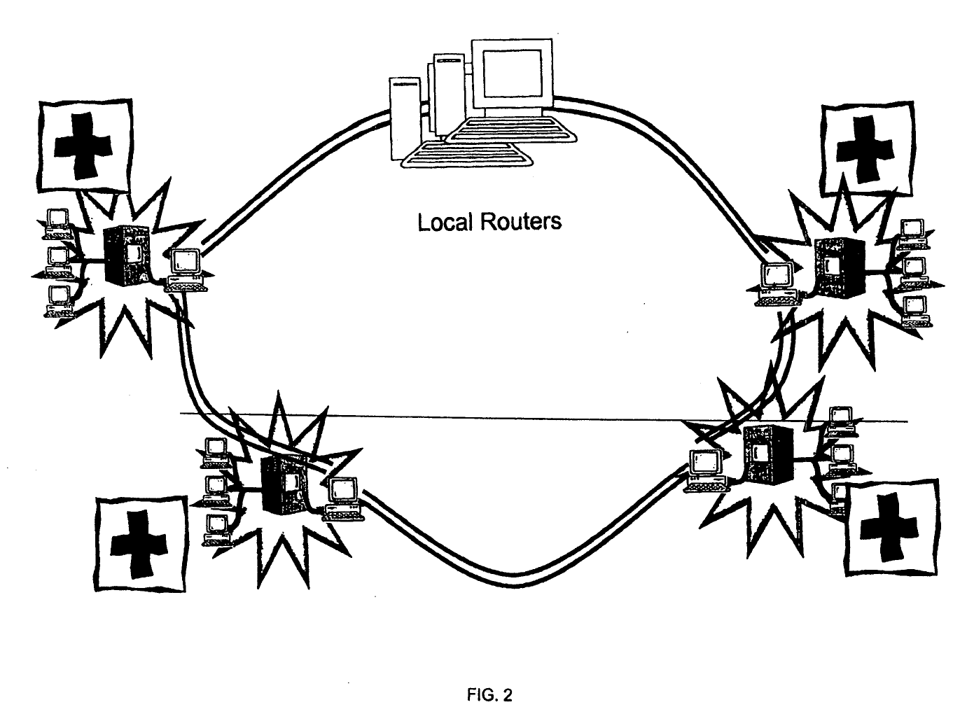

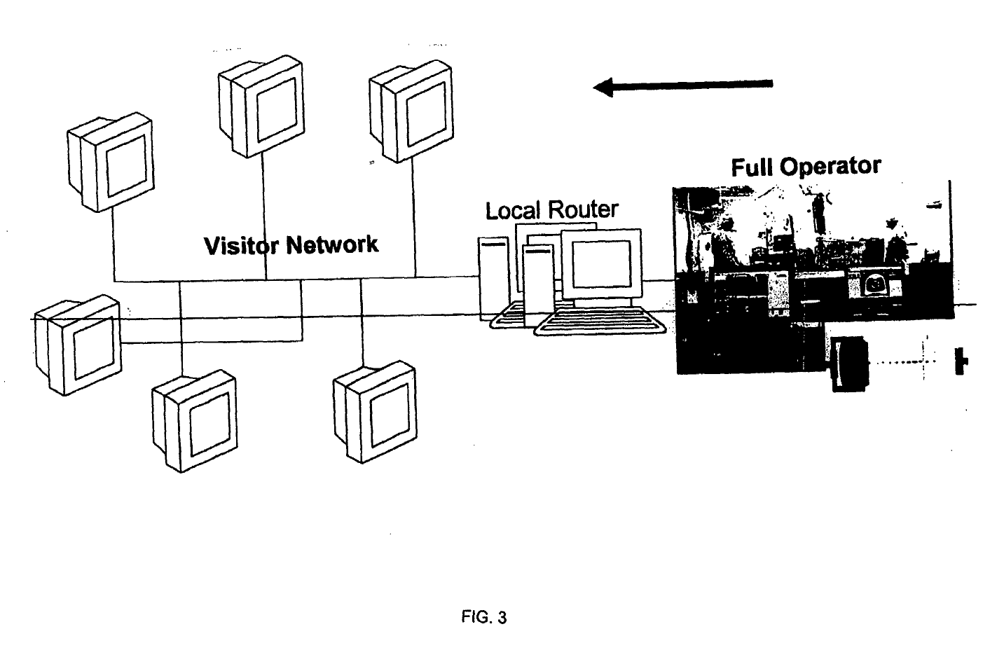

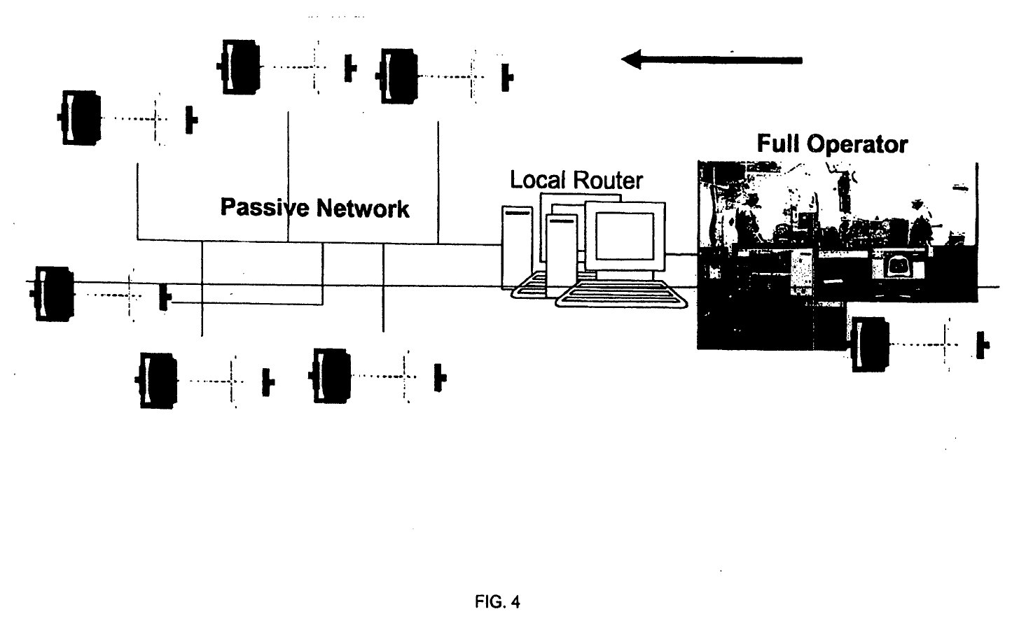

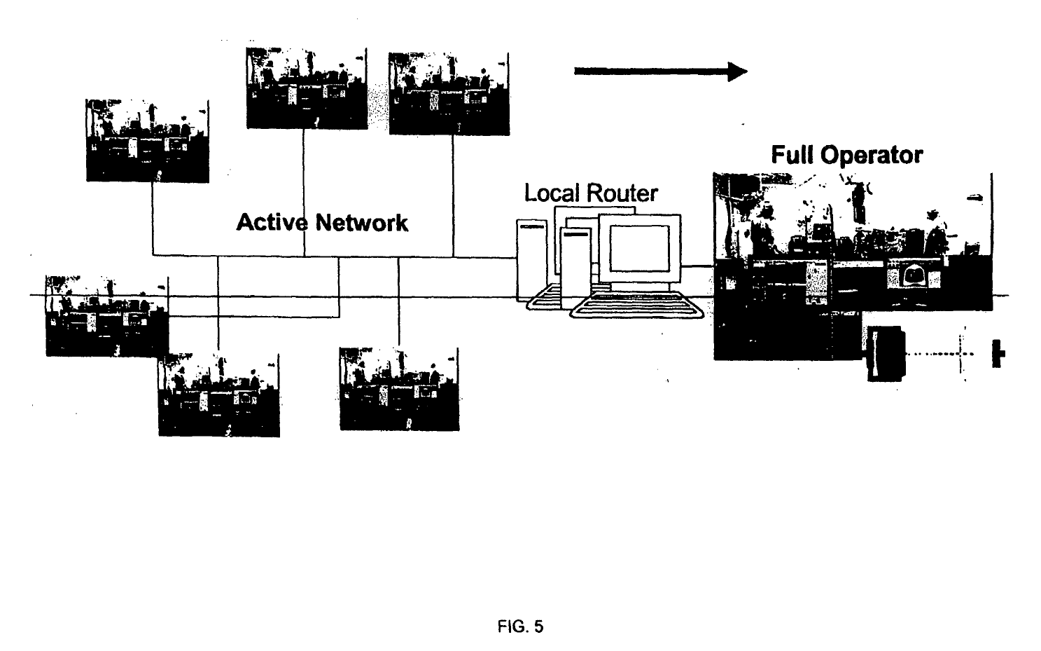

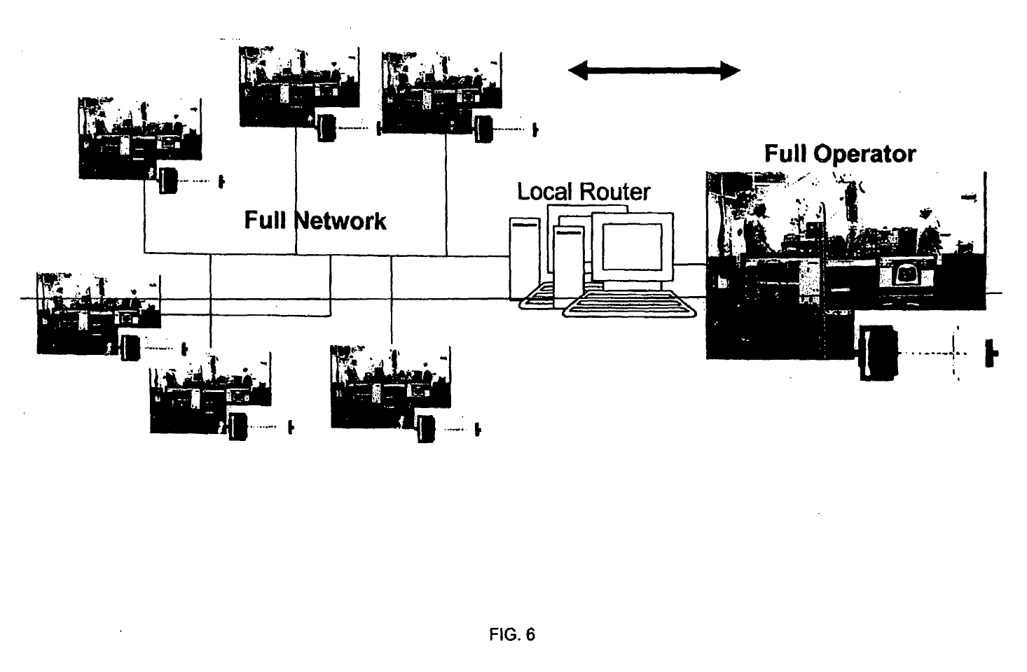

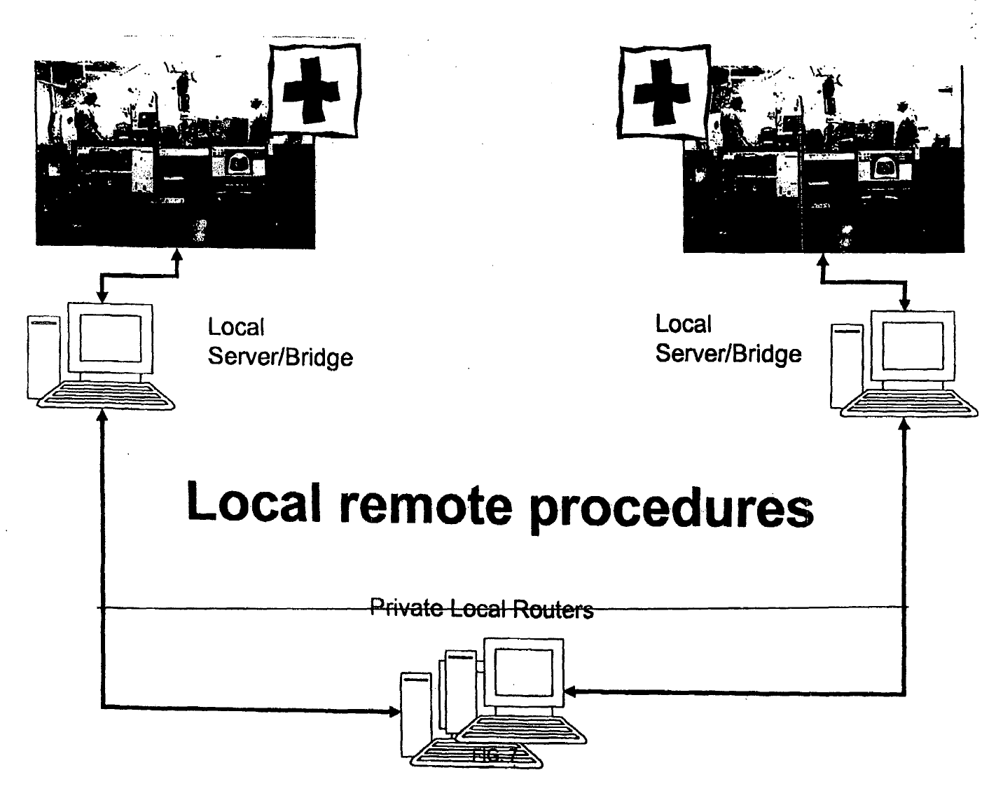

In one embodiment, a system is provided that comprises a local router that may be connected to one or more remote visitor, passive, active or full operator stations as shown in FIG. 2. The local router may be a double ring (active and idle ring) network in communication with servers at remote locations that have joined or connected to the local router. The local router is capable of acquiring the address of the remote operator location, and determining the operator type. For example, FIG. 3 shows a local router that is in communication with a plurality of remote Visitor operator stations, from which students or physicians may monitor or learn about a procedure being performed at a Full operator station via the system and network. Likewise, FIG. 4 shows a local router that is in communication with a plurality of remote Passive operator stations, from which physicians may watch or participate in a limited manner in a procedure being performed at a Full operator station. FIG. 5 shows a local router that is in communication with a plurality of Active operator stations and a Full operator station. From the Full operator station, a physician possessing expertise with such navigation systems can monitor several procedures being performed remotely at several Active operator stations. If an expert physician at the Full operator station determines that a certain remote procedure needs his assistance, the expert physician may use interface means at the Full operator station to control the navigation system at a remote Active operator station, and override the physician at the remote Active operator station. Thus, each patient at each remote Active operator station can receive the benefit of an expert physician supervising the medical procedure being performed. FIG. 6 shows a local router that is in communication with a plurality of Full operator stations and a central Full operator station. Such a network could also be implemented as a private network through a private local router and a plurality of Full operator stations as shown in FIG. 7.

The system further comprises a communication link that provides for communicating between the various surgical stations within the network. The communication link may be a physical communication means such as a fiber-optic communication channel, or alternatively may be a wireless communication means utilizing satellite communication for enabling surgeons to perform procedures from half way around the globe.

For enabling communication from the different sites in which to install different kind of workstations the best technologies to be used are optical fibers on a local basis and satellite connection on an international and intercontinental basis. On a local basis, a server should be installed in each site and a router directly interconnected with each local server. In this way a private and secure network can be set up to enable connections between sites. The communication links between sites optimally comprise fiber optic connection means. Among advantages of using optical fibers, the system achieves the greatest broadcast due to a reduced wavelength, signal frequency 1000 times more than satellite connections (speed*1000), and the highest C*P product (c, capacity of the system; p, repetition pass). Fiber optic communication provides up to 800 Gb/sec/km as compared to 10 and 1 Gb/sec/km for radio-based and coaxial wire-based connections,. Fiber-optic connections also provide the lowest attenuation of signal (0.4 dB/km), and allow for direct connections at great distances with a limited number of intermediate signal regenerators and immunity from electromagnetic interferences and safety from fulguration.

In some embodiments, a system is provided for enabling remote monitoring of a medical procedure being performed in a patient's body. The system comprises at least one full operator station having a navigation control system for controlling the orientation of a minimally interventional medical device that is to be guided within a subject body's anatomy, and one or more remote operator stations in communication with the at least one full operator station, wherein the medical procedure may be monitored from the one or more remote operator stations. The remote operator station may be a visitor operator station, from which a medical procedure may be monitored by a student or physician for providing education or training. The remote operator station may be a passive operator station, from which an operator may remotely participate in a limited capacity in a medical procedure being performed at a remote location. The remote operator station may be an active operator station, from which the operator may actively control the medical procedure that is to be performed at a remote location. The remote operator station may also be another full operator station. The network of remote operator stations are in communication with the at least one full operator station via a communication link and a local router. The communication link is preferably a fiber-optic communication means, but may alternatively be a wireless satellite communication link for enabling remote monitoring of a medical procedure that is being performed at a location that is at least part way around the earth.

In another aspect of the present invention, an active stations system is provided for enabling an operator to remotely perform a medical procedure in a patient's body at a remote location. The system comprises at least one full operator station having a navigation control system for controlling the orientation of a minimally interventional medical device that is to be guided within a subject body's anatomy, one or more remote operator stations in communication with the at least one full operator station, wherein the medical procedure may be controlled at least partially by an operator at the one or more remote operator stations. The one or more remote operator stations may be passive operator stations, from which an operator may remotely participate in a limited capacity in a medical procedure being performed at a remote location. The one or more remote operator stations may be active operator stations, from which an operator may actively control the medical procedure that is to be performed at a remote location. The remote operator station may also be another full operator station. Accordingly, a system and network of operator stations may be provided that provide for educational training, hands on training through remotely performing procedures in a limited capacity, and full control of a medical procedure from a remote location that may be a great distance from the patient and medical facility where the procedure is being conducted.

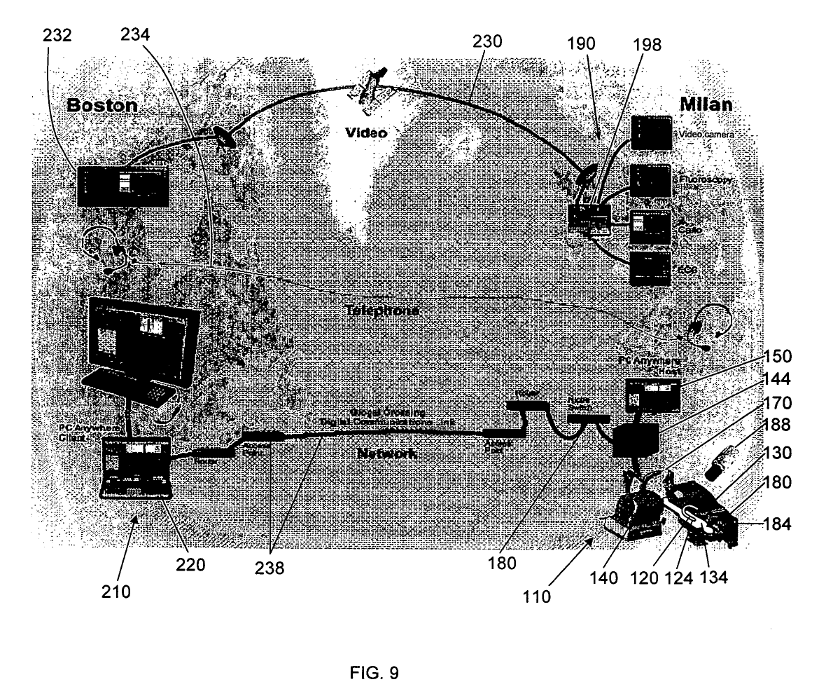

One example of a system for enabling an Active operator station for remotely performing surgical procedures on a patient who is geographically distanced from the performing physician is shown in FIG. 9. The system 100 comprises a computer-assisted navigational system 140 for directing and manipulating the distal tip of the medical device by remote actuation use computer assisted navigational systems. Computer-assisted navigational systems improve the control of such medical devices that contact tissues during surgical procedures, making these procedures more precise, repeatable and less dependent on the device manipulation skills of the physician. Computer-assisted navigational systems may also include an imaging system for providing imaging of the medical device and blood vessels and tissues. The system may also be configured to cooperate with a localization system. It is desirable to provide remote access to such a system from a potentially distant geographical location, among others in cases where a (distant) expert physician's knowledge and skills are useful in treating a patient's critical needs.

The system 100 provides for controlling a flexible medical device 120 in an operating region 130 in a subject's body 134 at a local procedure site 110, under the control of a user at a remote site 210. It should be noted that other remote systems may provide for control of different types of medical and surgical procedures. The system comprises a local navigation system 140 for selectively orienting the distal end 124 of the elongate medical device 120 in the operating region 130. The navigation system 140 includes a controller 144 responsive to control signals provided from a local computer 150. The system further includes a local device advancer (not shown) for advancing and retracting the device 120 in the operating region, which device advancer includes a controller (not shown) responsive to control signals provided from a local computer 150. The navigation system 140 may be a magnetic navigation system that applies a magnetic field to orient a magnetically responsive element 126 associated with the distal end 124 of the elongate medical device 120. The navigation system 140 may alternatively be a robotic system or an electrostrictive system that orients the distal end 124 of the elongate medical device 120.

The system further includes at least one local medical imaging system 170 for displaying an image of the operating region on a local display 172. The at least one medical imaging system 170 is preferably an X-ray or Fluoroscopic Imaging system, but may alternatively be a Magnetic Resonance imaging system or an ultrasound imaging system. A localization system 180 is included for determining the position of the medical device's distal end 124 in the localization system's own frame of reference, which is translatable to the local displayed image 172 of the local medical imaging system 170. The localization system's coordinate frame of reference is registered to the frame of reference of the imaging and navigation systems, such that localized medical device data is readily available for controlling navigation of the medical device 120 with the navigational system 140. The system further includes at least one subject physiology monitoring system 184, for monitoring the physiology of a subject patient and displaying information on local display 188 of the physiology monitoring system. Such a physiology monitoring system may be capable of measuring and displaying electrical activity of a tissue within the subject, or may be a system for monitoring the ElectroCardioGram (ECG) signal of the subject.

The system 100 further comprises at least one video imaging system 190 at the local procedure site 110, which is configured to display the image obtained from at least one camera. The video imaging system 190 may include a camera 188 for making a video image of the subject during the procedure. Alternatively, the camera 188 may be a mobile camera for making a video image of the procedure site, which may further be responsive to directions from a user at the remote site 200.

The system 100 further includes a local computer 150 for providing instructions from a local user to the navigation system controller 140 and the advancer controller 160. The system further includes at least one video imaging system 190 for providing video images of the local procedure site on a local display 198. The system also includes a remote site 210 having a remote computer 220 that allows a remote user to have access and input to the navigation system controller 144 and the advancer controller at the procedure site 110. A display device 232 is also included at the remote site 210.

The system 100 comprises at least three communication links between the local procedural site 110 and the remote site 210, which enable a user or physician at the remote site 210 to perform a medical procedure on a subject at the local procedural site 110. The communication links include a video linking system 230, an audio linking system 234 and a data linking system 238. The video linking system enables communication to the remote site of a video signal that provides a display of one or more of the images displayed by the various systems at the local procedure cite 110. The audio linking system 234 enables two-way communication between a user/physician at the remote cite and a user/physician at the local procedure cite, which two-way communication allows for coordinating the remotely performed procedure. Finally, the data linking system 238 provides for transmission of data signals from a remote computer 220 at the remote location 210 to the controller of the navigational system 140 at the local procedure cite 110, which data signals allow a user at the remote location 110 to control the navigation system to guide the medical device 120 within the subject 134 at the local procedure cite 110. Each of these communication links and their operation will be described in further detail below.

The system 100 comprises a video linking system 230 for providing a one-way communication of a combined video display signal on the display device 232 at the remote site 210. The combined video display combines at least two of the images being displayed at the local display 172 of the at least one local medical imaging system 170, and the images being displayed at the local display 198 of the at least one local video imaging system 190. The combined video display signal may further include the images being displayed on the local display 188 of the at least one subject physiology monitoring system 184. The video signal is typical of that used for a CRT-type monitor, such that the video signal contains much less signal information than the actual image data being processed for display by the imaging system 170, localization system 180, and physiological monitoring system 184. The video signal provides the same resolution as that being displayed at the local procedure cite. Accordingly, the video linking system provides for improved communication of displayed images, by transmitting video image data rather than the data used to generate the images. Moreover, the video linking system combines two or more of the images being displayed by the various display devices at the local procedure cite into one video signal, which allows for these images to be displayed on a single video display at the remote location, which reduces the need for duplicative display equipment.

The system 100 also comprises an audio linking system 234 for providing two-way audio communication between the local procedure site 110 and the remote site 210. This permits two-way audio communication, such as a telephone link, between a user/physician at the remote cite and a user/physician at the local procedure cite, which allows for coordinating the remotely performed procedure.

The system 100 further comprises a data linking system 238 for providing data communication between a computer 220 at the remote site 210 and the navigation system controller 144 and the advancer system controller. The data linking system allows a remote physician at a remote site 210 to provide inputs to the navigation system controller 144 for guiding the medical device's distal end 124 through the subject's body 134.

It should be noted that the computer 150 at the local procedure cite, or the controller of the navigation system 140, may be configured to give priority to commands from a user at the local site entered on the computer 150 at the local site 1 10, or to the controller 144 of the navigation system 140, such that the user at the local site 110 has priority control to implement the control signals sent by the remote user via the remote computer 220. In this manner, the physician at the remote location 210 could send a command to ablate a path of tissue on a subject 134 beginning at a certain point in the subject's electrocardiograph rhythm and ending after completing a given ablation path, and the local physician could implement the command from the local site 110. This would ensure that the transmission delay caused by significant distances separating the remote and local cites does not cause unwanted movements or ablation of the subject, and provides an added level of safety.

In another aspect of the present disclosure, various embodiments of a method may be provided for navigating an elongate medical device in an operating region in a subject's body at a local procedure site, by a user at a remote site. In one embodiment, a method is provided that comprises displaying on a display at the remote site a combined video image of one or more images being displayed at the local procedure site. The combined video image may include the local display of at least one local medical imaging system 170 which displays an image of the operating region on a local display 170, the local display of at least one video imaging system 190 that provides video images of the local procedure site on a local display 198; and the local display of at least one subject physiology monitoring system 184 that displays information about the subject's physiology on a local display.

The first embodiment of a method includes providing two way audio communication between the remote site and the local site for communication between the user at the remote site and the local site. The method further includes communicating commands from the user at the remote site entered on a computer at the remote site to a controller for controlling a navigation system at the local site 110 for operating the navigation system to selectively orient the distal end of the elongate medical device in the operating region, and communicating commands from the user at the remote site entered on a computer at the remote site to a controller for controlling a local device advancer for advancing and retracting the elongate medical device in the operating region. The method also communicates commands from a user at the local site entered on a computer at the local site to the navigation system having a controller responsive to control signals provided from a computer.

The method may further comprise prioritizing commands from a user at the local site entered on a computer at the local site over commands from a remote user entered on a remote computer, to provide for control of the navigation system and the advancer system. The user at the local site accordingly has priority control to implement command or control signals sent by the remote user via the remote computer 220. In this manner, the physician at the remote location could send a command to ablate a path of tissue on a subject beginning at a certain point in the subject's electrocardiograph rhythm and ending after completing a given path, and the local physician could implement the command. This would ensure that the transmission delay caused by significant distances separating the remote and local cites does not cause unwanted movements or ablation of the subject, and provides an added level of safety.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A system for navigating a medical device in an operating region in a subject's body at a local procedure site, under the control of a user at a remote site, the system comprising:
a local navigation system for selectively orienting the distal end of the elongate flexible medical device in the operating region, the navigation system including a controller responsive to control signals provided from a computer;
a local device advancer for advancing and retracting the device in the operating region, the device advancer including a controller responsive to control signals provided from a computer;
at least one local medical imaging system for displaying an image of the operating region on a local display;
at least one video camera imaging system for providing video images of the local procedure site on a local display;
at least one subject physiology monitoring system for displaying information about the subject's physiology on a local display;
a local computer for providing instructions from a local user to the navigation system controller and the advancer controller;
a remote computer for providing instructions from a remote user to the navigation system controller and the advancer controller;
a display at the remote site;
a video linking system for providing a combined video display on the display at the remote site of the local display of the at least one local medical imaging system, the local display of the at least one local video imaging system, and the local display of the at least one subject physiology monitoring system;
an audio linking system for providing two way audio communication between the local procedure site and the remote site; and
a data linking system for providing data communication between a computer at the remote site and the navigation system controller and the advancer system controller, wherein the data linking system communicates signals input by a user of the computer at the remote site for remotely controlling operation of the navigation system at the local site.

2. The system of claim 1, wherein the navigation system is configured to give priority to control signals received from the local controller from a local user over control signals received from the remote computer via the data linking system.

3. The system of claim 1, wherein the navigation system is configured to execute the control signals received from the remote computer via the data linking system upon authorization from a local user at the local site.

4. The system according to claim 1 wherein the at least one medical imaging system is one of the group consisting of a fluoroscopic imaging system, a magnetic resonance imaging system, and an ultrasound imaging system.

5. The system according to claim 1 wherein the at least one subject physiology system is a system for measuring and displaying electrical activity.

6. The system according to claim 1 wherein the at least one subject physiology system is a system for measuring and displaying an electrocardiogram.

7. The system according to claim 1 wherein the at least one video imaging system includes one of a stationary camera for making a video image of the subject during the procedure, or a mobile camera for making a video image of the procedure site responsive to directions from a user at the remote site.

8. The system according to claim 1 wherein the navigation system is one of a magnetic navigation system that applies a magnetic field to orient a magnetically responsive element associated with the distal end of the elongate medical device, a robotic system that orients the distal end of the elongate medical device, or an electrostrictive system that orients the distal end of the elongate medical device.

9. A system for navigating a medical device in an operating region in a subject's body at a local procedure site, under the control of a user at a remote site, the system comprising:
a navigation system at the local site for controllably navigating the medical device in the operating region within the subject, the navigation system including a controller responsive to control signals provided from a computer, for applying a magnetic field to selectively orienting the distal end of the flexible medical device in a desired direction;
a local device advancer for advancing and retracting the device in the operating region, the device advancer including a controller responsive to control signals provided from a computer;
at least one local fluoroscopy imaging system for displaying an image of the operating region on a local display;
at least one video camera imaging system for providing video images of the local procedure site on a local display;
at least one subject physiology monitoring system for displaying information about the subject's physiology on a local display;
a local computer for providing instructions from a local user to the navigation system controller and the advancer controller;
a remote computer at a remote site for providing instructions from a remote user to the navigation system controller and the advancer controller;
a display at the remote site;
a video linking system for providing on the display at the remote site a combined video display including the video image of the at least one fluoroscopy imaging system, the video image of the at least one video camera imaging system display, and the video image of the at least one subject physiology monitoring system;
an audio linking system for providing two way audio communication between the local procedure site and the remote site; and
a data linking system for providing data communication between a remote computer at the remote site and the navigation system controller and the advancer system controller, wherein the data linking system communicates signals input by a user of the remote computer at the remote site for remotely controlling operation of the navigation system at the local site.

10. The system of claim 9 wherein the at least one video imaging system includes one of a stationary camera for making a video image of the subject during the procedure, or a mobile camera for making a video image of the procedure site responsive to directions from a user at the remote site.

11. The system according to claim 9 wherein the at least one subject physiology system is a system for measuring and displaying an electrocardiogram.

12. The system of claim 9 wherein the video linking system receives video output signals from the at least one fluoroscopy imaging system, the at least one video camera imaging system, and the at least one subject physiology monitoring system, and is configured to generate a new video output signal for providing a combined display of the video display images from the at least one fluoroscopy imaging system, the at least one video camera imaging system, and the at least one subject physiology monitoring system.

13. The system of claim 9 wherein the navigation system is configured to give priority to control signals received from the local controller from a local user over control signals received from the remote computer via the data linking system.

14. The system of claim 11 wherein the navigation system is configured to execute the control signals received from the remote computer via the data linking system upon authorization from a local user at the local site.

15. The system according to claim 14 wherein the control signals received from the remote computer are executed at a point in time relative to the electrocardiogram of the subject.

16. A method of navigating an elongate flexible medical device in an operating region in a subject's body at a local procedure site, by a user at a remote site, the method comprising the steps of:
displaying on a display at the remote site a combined video image of the local display of at least one local medical imaging system that displays an image of the operating region on a local display; the local display of at least one video imaging system that provides video images of the local procedure site on a local display; and the local display of at least one subject physiology monitoring system that displays information about the subject's physiology on a local display;
providing two way audio communication between the remote site and the local site for communication between the user at the remote site and the local site;
communicating commands from the user at the remote site entered on a computer at the remote site to a controller for controlling a navigation system at the local site for operating the navigation system to selectively orienting the distal end of the elongate medical device in the operating region; and
communicating commands from the user at the remote site entered on a computer at the remote site to a controller for controlling a local device advancer for advancing and retracting the elongate medical device in the operating region

17. The method of claim 16 further comprising the step of communicating commands from a user at the local site entered on a computer at the local site, for controlling the navigation system and the advancer system.

18. The method of claim 17 wherein the navigation system is configured to give priority to command signals received from a user at the local site over command signals received from the remote computer at the remote site.

19. The system of claim 16 wherein the navigation system is configured to execute the command signals received from the remote site upon authorization from a user at the local site.

20. The method of claim 19 wherein the at least one subject physiology system is a system for measuring and displaying an electrocardiogram, and the command signals received from the remote site are executed at a time relative to the electrocardiogram of the subject.
